(19) United States
(12) Patent Application Publication
(10) Pub. No.: US 2026/0263271 A1
(43) Pub. Date: Sep. 10, 2026

(54) WIDE ANGLE VIEWING SYSTEM WITH OPHTHALMIC ILLUMINATION SWITCHING FUNCTIONALITY

(71) Applicant: Alcon Inc., Fribourg (CH)

(72) Inventor: David Chu, Goleta, CA (US)

(21) Appl. No.: 19/545,694

(22) Filed: Feb. 20, 2026

Related U.S. Application Data
(60) Provisional application No. 63/768,031, filed on Mar. 6, 2025.

(51) Int. Cl. A61F 9/007; A61B 17/00; A61B 34/20; A61B 90/20; A61B 90/30; G16H 40/67

(52) U.S. Cl. CPC A61F 9/007; A61B 34/20; A61B 90/20; A61B 90/30; G16H 40/67; A61B 2017/00199; A61B 2034/2055; A61B 2034/2059

(57) ABSTRACT

A wide angle viewing system (WAVS) includes a microscope having a microscope light source, a front lens assembly connected to the microscope that is moveable between engaged and disengaged positions, and a processor. The processor performs a method during which the processor determines a current position of the front lens assembly as one of the engaged or disengaged position and executes an illumination switching control action in response to the current position. The control action includes turning off the microscope light source and turning on the illumination tool when the front lens assembly is in the engaged position. The control action also includes turning on the microscope light source and turning off the illumination tool when the front lens assembly is in the disengaged position.

### INTRODUCTION

The present disclosure relates to a wide angle viewing system (WAVS) having a microscope-attached front lens assembly for viewing a patient's ocular anatomy, e.g., during vitreoretinal surgery. A typical vitreoretinal surgery involves the performance of delicate surgical tasks in and around the fundus region of the eye. The accurate prognosis and diagnosis of injury, disease, and other conditions of the eye often requires a surgeon to use a microscope to view the eye under high levels of magnification. Image capture capabilities may be provided by a microscope-mounted digital camera. In this manner, the surgeon is afforded a clear view of the retina, macula, vitreous humor, and surrounding tissue within the eye.

During an ophthalmic visualization procedure, a surgeon may require a wider view of the fundus region than is ordinarily achievable solely using the microscope's internal lenses. For instance, the surgeon might find it beneficial to view the peripheral retina area when monitoring for retinal tears or detachments. For this purpose, the above-noted WAVS includes a specially-constructed front lens, which in some implementations is placed directly on the patient's cornea as a contact lens. In contrast to a contact-type WAVS, an indirect/non-contact WAVS positions the front lens several millimeters away from the patient's cornea. The front lens in either instance provides the surgeon with a wide angle view of the fundus region.

### SUMMARY

Disclosed herein are systems and methods for automatically switching between different ophthalmic lighting sources during a vitreoretinal surgery or visualization process. The lighting sources considered herein include a microscope-mounted lamp or another application suitable microscope-based lighting source for illuminating a patient's eye from outside the eye, and an illumination tool, e.g., an endoilluminator or a chandelier insertable into the patient's eye, with the latter devices being configured for illuminating the patient's eye from within.

In particular, a wide angle viewing system (WAVS) as described herein includes an ophthalmic microscope having a microscope light source, e.g., a xenon lamp, a light emitting diode (LED) array, etc. The WAVS includes or is in communication with a surgical console, with the above-noted illumination tool and possibly other surgical tools being connected to and powered by the surgical console. The WAVS further includes a reduction lens assembly and an adjustable front lens assembly. The reduction lens assembly is connected to the microscope, with the front lens assembly connected in turn to the reduction lens assembly.

The adjustable front lens assembly is moveable between distinct engaged and disengaged positions. A processor of the microscope, which is in communication with the microscope light source and with an illumination tool, is configured to determine a position of the front lens assembly as being the engaged position or the disengaged position. In one or more embodiments, the engaged/disengaged position may be determined by sensing the position of the front lens assembly. In other embodiments, the position is inferred from surgical stage information communicated to the processor by the surgical console. The processor also executes an illumination switching control action in response to the engaged/disengaged position of the front lens assembly. The illumination switching control action in one or more implementations includes (i) turning off the (external) microscope light source and turning on the (internal) illumination tool when the front lens assembly is in the engaged position, and (ii) turning on the microscope light source and turning off the illumination tool when the front lens assembly is in the disengaged position.

The surgical console is connectable to and configured to energize the illumination tool. The processor is configured to transmit an electronic switching control signal to the surgical console to cause the surgical console to turn on the illumination tool when the front lens assembly is in the engaged position, and to turn off the illumination tool when the front lens assembly is in the disengaged position.

The WAVS may include at least one position sensor operable for sensing the current engaged/disengaged position of the front lens assembly and outputting an electronic position signal indicative of the current position. The processor may determine whether the front lens assembly is in the engaged or disengaged position by processing the electronic position signal, e.g., by comparing information in the signal to reference values indicative of the engaged or disengaged position.

An optional human-machine interface (HMI) device may be placed in communication with the processor and used to transmit an override signal to the processor in response to an operator input. In such an embodiment, the processor may interrupt the illumination switching control action in response to the override signal, thereafter entering a surgeon-controlled operating mode.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 illustrates an exemplary wide-angle visualization system (WAVS) configured to control illumination switching actions in accordance with the present disclosure.

FIG. 1A illustrates a portion of the WAVS of FIG. 1 when used during a representative vitreoretinal surgery.

FIG. 2 is a schematic block diagram illustrating operation of components of the WAVS shown in FIG. 1.

FIG. 3 is a flow chart describing a method for controlling an illumination state during a vitreoretinal surgery performed using the WAVS of FIG. 1.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

Embodiments of the present disclosure are described in detail herein. Disclosed embodiments are provided as examples, with other embodiments possibly taking alternative forms. The Figures are not necessarily drawn to scale. For instance, some features could be exaggerated or minimized to show details of particular components. Therefore, specific structural and functional details disclosed herein are not to be interpreted as limiting, but merely as a representative basis for teaching one skilled in the art to employ the present disclosure.

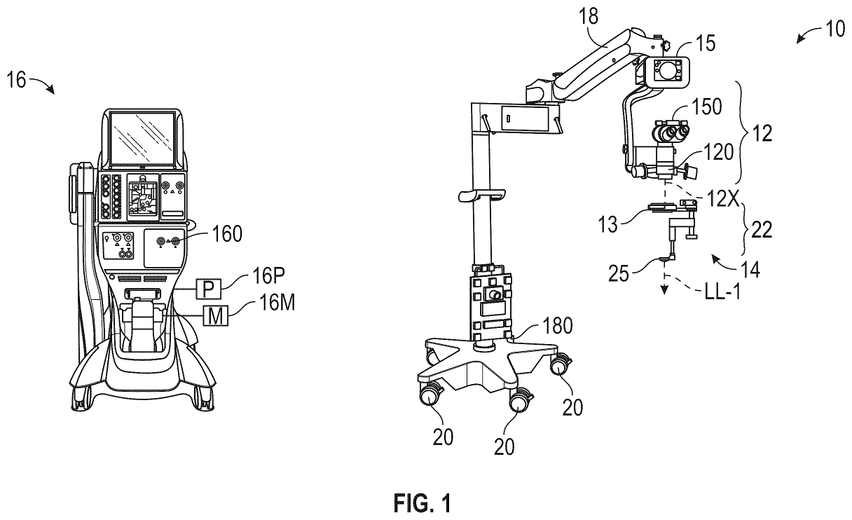

Referring to the drawings, wherein like reference numbers refer to like components, a wide angle viewing system (WAVS) 10 is illustrated in FIG. 1 that includes an ophthalmic microscope 12, a reduction lens assembly 13 connected to the microscope 12, and an adjustable front lens assembly 14 connected to the reduction lens assembly 13. The WAVS 10 in one or more embodiments may also include or communicate with a surgical console 16 and a support arm 18, with the support arm 18 being operatively connected to a base 180. For mobility within an operating room, the base 180 may be equipped with a set of lockable wheels 20 or other suitable appendages such as feet, rollers, etc.

In the illustrated configuration, the microscope 12, e.g., LuxOR® Revalia™, is connected to the support arm 18, the reduction lens assembly 13, and the front lens assembly 14, the latter being moveable between distinct engaged and disengaged positions as set forth below. The front lens assembly 14, which includes a magnifying front lens 25, is connected to the microscope 12 by virtue of being removably connectable to an intervening connecting portion 22, for instance a ZEISS Resight™ Fundus Imaging System or the OCULUS BIOM®. The microscope 12 includes a microscope light source 120. The microscope light source 120 for its part is configured to direct external light (LL-1) along an optical axis 12X of the microscope 12 and toward a patient's eye 26 (FIG. 1A). The microscope 12 or the WAVS 10 may also include digital oculars 150 and one or more high-resolution medical display screens 15 for viewing the eye 26 during a vitreoretinal surgery, as appreciated in the art.

Ophthalmic surgical consoles such as the representative surgical console 16 of FIG. 1 are typically equipped with multiple columns and rows of connection ports 160 providing the requisite electrical, data, pressure, irrigation, suction, and other power connections needed for supporting a given surgical procedure and its related surgical tools. The surgical console 16 is thus connectable to the illumination tool 30 as part of the present approach, and may be configured as or include an electronic control unit in communication with other systems or components, e.g., via a wired or wireless communications network or individual transfer conductors.

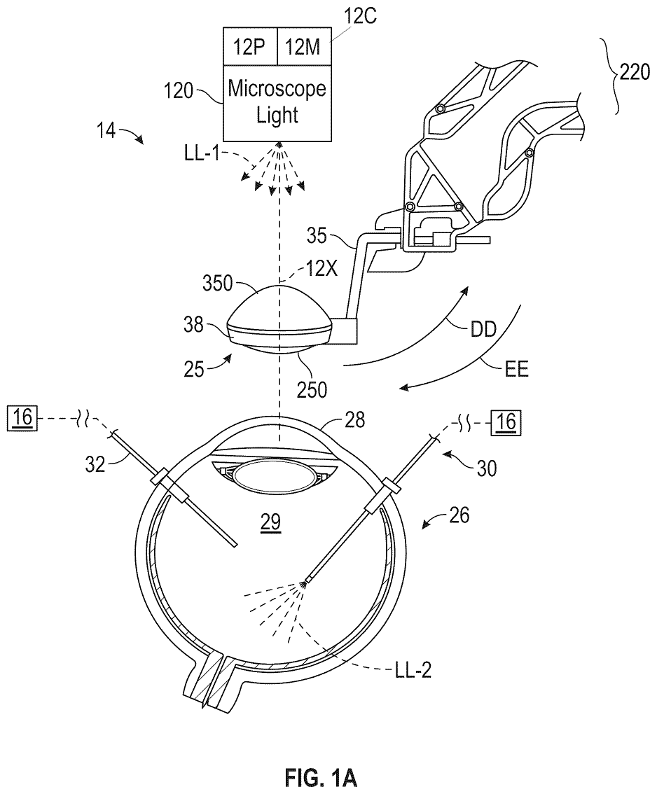

The surgical console 16 may also include one or more processors (P) 16P and sufficient computer-readable storage media/tangible non-transitory memory (M) 16M, e.g., optical, magnetic, flash, or other types of read only memory, along with application-sufficient amounts of random-access memory, electrically-erasable programmable read only memory, etc. The processor(s) for their part may be constructed from various combinations of Application Specific Integrated Circuit(s) (ASICs), Field-Programmable Gate Arrays (FPGAs), electronic circuits, central processing units, microprocessors, and the like. The processor 16P and the memory 16M may be used as a control node for the WAVS 10 as set forth herein, or portions of the described functionality may be programmed into another accessible memory location, e.g., a processor 12P and memory 12M of the microscope 12 as shown in FIGS. 1A and 2, which together may form a control system 12C.

Referring to FIG. 1A, the processor 12P of the microscope 12 in a possible embodiment is connected to a computer readable storage medium (“memory”) 12M of the microscope 12, and is in communication with the microscope light source 120 and an illumination tool 30 (FIG. 1A). The illumination tool 30 may be embodied as an endoilluminator or a chandelier configured to emit internal light LL-2, with “external” and “internal” with respect to respective light LL-1 and LL-2 referring to the origin of the light relative to the eye 26. The eye 26 defines a vitreous cavity 29 as it appears while undergoing a vitrectomy surgery, with wide angle viewing assistance provided by the WAVS 10 (FIG. 1). The illumination tool 30 and a representative surgical tool 32, e.g., a vitrector, both penetrate the eye 26 through a respective cannula and are manipulated therein to perform an operation within the vitreous cavity 29. Control settings of the illumination tool 30 and the surgical tool 32 are both connected to and controlled by the surgical console 16.

The front lens 25 of the front lens assembly 14, which has respective lower and upper convex surfaces 250 and 350, is connected to a bracket 35 via a connecting loop 38. When wide angle viewing via the front lens 25 is desired, the surgeon moves the front lens assembly 14 to position the front lens 25 in the optical axis 12X of the microscope 12, as indicated by arrow EE. Movement may occur via articulation, pivoting, rotation, or other movement of the connecting portion 22 of FIG. 1, shown in FIG. 1A as an alternatively constructed latticed connecting portion 220. During surgery or in-office visualization, the lower surface 250 of the front lens 25 is moved to a predetermined standoff distance from a cornea surface 28 of the eye 26, with a standoff distance of about 5-10 millimeters (mm) being typical. The surgeon may move the front lens assembly 14 and its front lens 25 out of the optical axis 12X in the opposite direction (arrow DD) when wide angle viewing via the front lens 25 is not required. The front lens 25 is therefore “non-contact” in the non-limiting embodiment of FIG. 1A, in the sense that the front lens 25 does not physically contact the cornea surface 28, contrary to a contact-type WAVS. However, the present teachings may be applied to contact-type WAVS embodiments within the scope of the disclosure.

LIGHTING CONTROL: The processor 12P or another suitable processor of the WAVS 10 shown in FIG. 1 is configured to determine a current position of the front lens assembly 14 as being one of the engaged position or the disengaged position. As noted above, the current position may be sensed or inferred. With respect to the latter option, the processor 12P may infer the position based on a reported surgical stage. In a typical cataract procedure, for instance, a surgeon performs a number of discrete steps: incision, capsulorhexis, lens chop, aspiration, lens polish, aspiration, intraocular lens (IOL) delivery, IOL rotation, etc. As another example, in a vitreous/retinal procedure, example steps may include: creating entry ports, vitreous removal, retinal treatment, filling of eye, closing incision, etc. It will be appreciated that similar steps or stages may be observed or detected for any ophthalmic surgery or procedure. The surgical console 16 of FIG. 1 may communicate such surgical stage information to the processor 12P, e.g., by transmitting a stage signal wirelessly or via physical transfer conductors. In a possible implementation, the engagement, disengagement, illumination ON, illumination OFF, and other states or settings may be controlled in response to the reported stage information from the surgical console 16.

As used herein, the engaged position is one in which the front lens 25 has been moved in the direction of arrow EE such that the front lens 25 is centered on the optical axis 12X. An exemplary engaged position is shown in FIG. 1A. In contrast, the disengaged position is achieved when the front lens 25 has been moved out of the optical axis 12X in the direction of arrow DD, as noted above.

The processor 12P is programmed herein to execute an illumination switching control action in response to the position of the front lens assembly 14, whether sensed or inferred from a reported surgical stage. The illumination switching control action in one or more embodiments includes turning off the microscope light source 120 and turning on the illumination tool 30 when the front lens assembly 14 is in the engaged position. The eye 26 of FIG. 1A is illuminated from within via the light LL-2 in this instance. Likewise, the processor 12P is configured to turn on the microscope light source 120 and turn off the illumination tool 30 when the front lens assembly 14 is in the disengaged position. In this mode, the eye 26 is externally illuminated via the light LL-1 from the microscope light source 120.

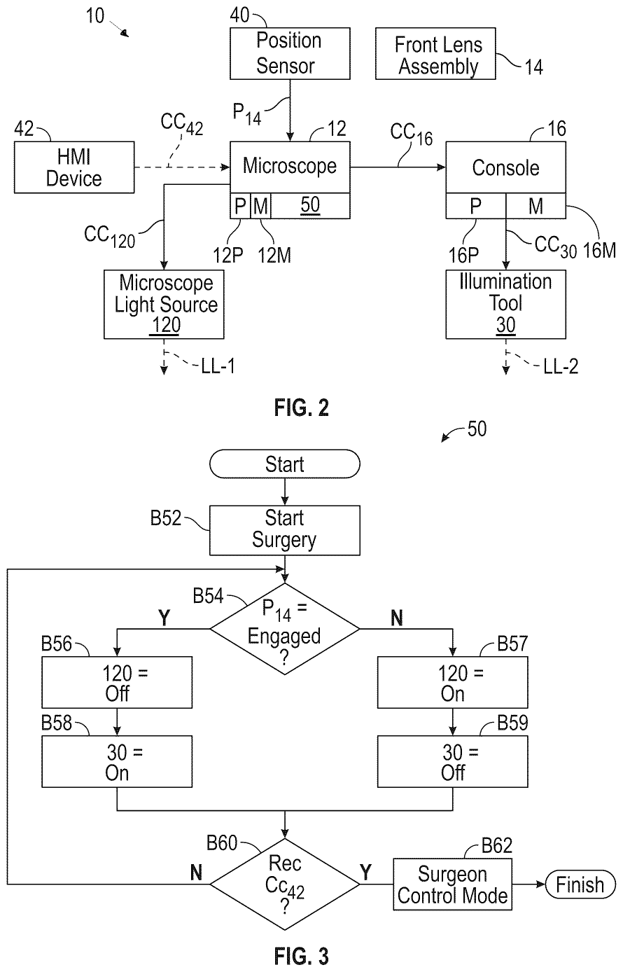

Referring now to FIG. 2, the processor 12P of the microscope 12 in one or more embodiments is configured to execute instructions from memory 12M embodying a method 50, an exemplary embodiment of which is shown in FIG. 3 and discussed below. The processor 12P is configured to transmit an electronic switching control signal (CC₁₆) to the surgical console 16 to cause the processor 16P of the surgical console 16 to turn on the illumination tool 30 so that light (LL-2) is emitted via the illumination tool 30 when the front lens assembly 14 is in the engaged position, and so that the surgical console 16 turns off illumination tool 30 when the front lens assembly 14 is in the disengaged position. The surgical console 16 in turn is connectable to the illumination tool 30 and configured to energize the illumination tool 30, e.g., via an illumination control signal (CC₃₀). The processor 12P of microscope 12 is also configured to transmit an electronic switching control signal (CC₁₂₀) to the microscope light source 120 to cause the microscope light source 120 to turn on and emit the light (LL-1) when the front lens assembly 14 is in the disengaged position, and to cause the microscope light source 120 to turn off/stop emitting the light (LL-1) when the front lens assembly 14 is in the engaged position.

As part of the present approach, a position sensor 40 of the WAVS 10 in non-limiting embodiments is operable for determining a current position of the front lens assembly 14 and outputting an electronic position signal (P₁₄) indicative of the current position. The processor 12P in this particular construction is configured to determine whether the front lens assembly 14 is in the engaged or disengaged position by processing the electronic position signal (P₁₄), i.e., the current position of the front lens assembly 14. For instance, the processor 12P may compare information in the position signal (P₁₄) to previously recorded information indicative of the engaged and disengaged positions. The processor 12P may then command the surgical console 16 to control an on/off or other light setting of the illumination tool 30 in response to the current position.

The position sensor 40 in accordance with various constructions may include, e.g., a simple on/off switch having a corresponding binary state such as “1” for engaged/on and “0” for disengaged/off, or a resolver or rotary encoder coupled to the front lens assembly 14 and configured to sense the position of the front lens assembly 14, and output the electronic position signal (P₁₄) as an encoder signal, e.g., a sine-cosine signal as appreciated in the art. Alternatively, the position sensor 40 may be positioned remotely from the front lens assembly 14, i.e., not mechanically coupled to the front lens assembly 14 or not connected to the front lens assembly. Such a remote sensor may include a camera, for instance, with the processor 12P in such an implementation being configured to process the electronic position signal (P₁₄) as an output signal from the remote sensor, e.g., as image data in the representative case of the camera, using computer vision software, pattern recognition, a neural network, etc., to detect the position of the front lens assembly 14. Other possible implementations of the position sensor 40 may be contemplated by those skilled in the art.

In an optional construction, a human-machine interface (HMI) device 42 is in communication with the processor 12P and configured to transmit an optional override signal (CC₄₂) to the processor 12P in response to an operator input, e.g., a surgeon's touch or voice input to the HMI device 42. The processor 12P in such an embodiment is configured to interrupt the illumination switching control action in response to receipt of the override signal (CC₄₂).

Referring to FIG. 3, the method 50 is described in terms of discrete process steps, segments, or logic blocks for clarity. Some of the hardware solutions set forth above may be implemented in software, for example by programming the memory 12M of the microscope 12 shown in FIGS. 1 and 1A with computer-readable instructions, algorithms/code segments, or logic blocks, the execution of which by the processor 12P causes the processor 12P to perform the noted control actions. Thus, a corresponding automated routine may initialize (“Start”) with commencement of a vitrectomy or other surgical/visualization procedure of the eye 26 shown in FIG. 1A.

In general, method 50 for controlling the WAVS 10 of FIGS. 1 and 1A includes determining a current position of the front lens assembly 14 of the WAVS 10 as one of an engaged position or a disengaged position. The method 50 includes executing an illumination switching control action in response to the current position of the front lens assembly 14, with the illumination switching control action including (1) turning off a microscope light source 120 of the microscope 12 and turning on the illumination tool 30 when the front lens assembly 14 is in the engaged position, and (2) turning on the microscope light source 120 and turning off the illumination tool 30 when the front lens assembly 14 is in the disengaged position.

The method 50 in the non-limiting implementation of FIG. 3 includes initializing the microscope 12 and the surgical console 16 of FIG. 1. Thereafter, a core algorithm for method 50 may load for execution by processor 12P. The method 50 proceeds to block B52.

At block B52 (“Start Surgery”), the surgeon commences performance of the vitreoretinal surgery using the surgeon's preferred source of illumination, i.e., the microscope light source 120 or the illumination tool 30. The method 50 thereafter proceeds to block B54.

Block B54 (“P₁₄=Engaged?”) includes determining whether the front lens assembly 14 is in the engaged position. Block B54 may entail receiving and processing the position signal (P₁₄) from the position sensor 40 (FIG. 2) via the processor 12P, with the position signal (P₁₄) representing the engaged/disengaged position of the front lens assembly 14. Depending on the configuration of the position sensor 40, information conveyed in the position signal (P₁₄) may vary, e.g., as a voltage indicative of the current position, as a binary signal when the position sensor 40 is implemented as a simple switch, as image data when the position sensor 40 includes a camera, etc. Other embodiments of block B54 may include inferring the position based on the reported surgical stage. The method 50 proceeds to block B56 when the processor 12P has determined that the front lens assembly 14 is in the engaged position of FIG. 1A, i.e., the front lens 25 is in the optical axis 12X. The method 50 proceeds in the alternative to block B57 when the processor 12P has determined that the front lens assembly 14 is in the disengaged position.

At block B56 (“120=Off”), the processor 12P may command the microscope light source 120 to turn off as a control response when the front lens assembly 14 is in the engaged position of FIG. 1A. The method 50 proceeds to block B58 after turning off the microscope light source 120.

At block B57 (“120=On), the processor 12P may command the microscope light source 120 to turn on as a control response when the front lens assembly 14 is in the disengaged position, i.e., when the front lens 25 of FIG. 1A is moved out of the optical axis 12X in the direction of arrow DD. The method 50 thereafter proceeds to block B59.

At block B58 (“30=On”), the processor 12P next commands the illumination tool 30 (FIG. 1A) to turn on as a control response when the front lens assembly 14 is in the engaged position. The method 50 then proceeds to block B60.

Block B59 (“30=Off”) includes commanding the illumination tool 30 (FIG. 1A) to turn off as a control response when the front lens assembly 14 is in the disengaged position. The method 50 then proceeds to block B60.

At block B60 (“Rec CC₄₂?”), the processor 12P determines whether the optional override signal (CC₄₂) of FIG. 2 has been received from the HMI device 42. As noted above, the override signal (CC₄₂) may be generated when the surgeon requests manual control of the on/off states of the microscope light source 120 and the illumination tool 30. This may occur at any point of the method 50, and therefore block B62 is shown in just one possible loop location in FIG. 3. The method 50 proceeds to block B62 when the override signal (CC₄₂) is received or detected, with the method 50 returning to block B54 when the override signal (CC₄₂) is not received or detected.

Block B62 (“Surgeon Control Mode”) may be performed as a control action in response to an affirmative decision at block B60, i.e., when the processor 12P detects the override signal (CC₄₂). The method 50 is finished when the surgeon assumes control of the lighting states. In some implementations, block B62 may be performed in a continuous loop with block B60 concurrently with the rest of the method 50 to allow the surgeon to override automatic illumination switching control at any point of the surgery.

The present solutions thus control the on/off states of different illumination sources during a vitreoretinal surgery while maintaining an approach for establishing surgeon control over the switching decision. Surgery time is reduced and potential complications are avoided by eliminating the need for the surgeon to turn off one lighting source and turn on the other when moving the front lens assembly 14 of FIG. 1A (or a contact-type alternative) into or out of the optical axis. These and other benefits of the present disclosure will be readily appreciated by those skilled in the art, now having the benefit of the foregoing disclosure.

As those of ordinary skill in the art will understand, various features illustrated and described with reference to any one of the Figures can be combined with features illustrated in one or more other Figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Embodiments shown in the drawings or the characteristics of various embodiments mentioned in the present description are not necessarily to be understood as being independent of each other. It is possible that each of the characteristics described in a given embodiment could be combined with one or more other desired characteristics from other embodiments, resulting in other embodiments not described in words or by reference to the drawings. Accordingly, such other embodiments fall within the framework of the scope of the appended claims.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

We claim:

1. A wide angle viewing system (WAVS), comprising:
an ophthalmic microscope having a microscope light source;
a front lens assembly connected to the microscope, the front lens assembly being moveable between an engaged position and a disengaged position; and
a processor in communication with the microscope light source and with an illumination tool, wherein the processor is configured to:
determine a current position of the front lens assembly as one of an engaged position or a disengaged position; and
execute an illumination switching control action in response to the current position of the front lens assembly, the illumination switching control action including:
turning off the microscope light source and turning on the illumination tool when the front lens assembly is in the engaged position; and
turning on the microscope light source and turning off the illumination tool when the front lens assembly is in the disengaged position.

2. The WAVS of claim 1, further comprising:
a surgical console connectable to the illumination tool, wherein the processor is configured to transmit an electronic switching control signal to the surgical console to command the surgical console to turn on the illumination tool in the engaged position and turn off the illumination tool in the disengaged position.

3. The WAVS of claim 1, further comprising:
a position sensor operable for determining the current position of the front lens assembly and outputting an electronic position signal indicative of the current position, wherein the processor is configured to determine whether the front lens assembly is in the engaged position or the disengaged position by processing the electronic position signal.

4. The WAVS of claim 3, wherein the position sensor includes an encoder that is coupled to the front lens assembly.

5. The WAVS of claim 3, wherein the position sensor includes a remote sensor that is not mechanically coupled to the front lens assembly.

6. The WAVS of claim 5, wherein the remote sensor includes a camera, and wherein the processor is configured to process image data from the camera to detect the current position of the front lens assembly.

7. The WAVS of claim 1, further comprising:
a human-machine interface (HMI) device in communication with the processor, the HMI device being configured to transmit an override signal to the processor in response to an operator input, wherein the processor is configured to interrupt the illumination switching control action in response to the override signal.

8. A control system for a wide angle viewing system (WAVS), comprising:
a processor in communication with a microscope light source and with an illumination tool; and
a computer readable storage medium (“memory”) on which is recorded instructions, the instructions being executable by the processor to cause the processor to:
determine a current position of a front lens assembly of the WAVS as one of an engaged position or a disengaged position; and
execute an illumination switching control action in response to the current position of the front lens assembly, the illumination switching control action including:
turning off the microscope light source and turning on the illumination tool when the front lens assembly is in the engaged position; and
turning on the microscope light source and turning off the illumination tool when the front lens assembly is in the disengaged position.

9. The control system of claim 8, wherein execution of the instructions causes the processor to:
transmit an electronic switching control signal to a surgical console to cause the surgical console to turn on the illumination tool in the engaged position and turn off the illumination tool in the disengaged position.

10. The control system of claim 8, wherein execution of the instructions causes the processor to:
receive an electronic position signal from a position sensor, the electronic position signal being indicative of the current position of the front lens assembly; and
determine whether the front lens assembly is in the engaged position or the disengaged position by processing the electronic position signal.

11. The control system of claim 10, wherein the position sensor includes an encoder that is coupled to the front lens assembly, and wherein execution of the instructions causes the processor to receive the electronic position signal as an encoder signal from the encoder.

12. The control system of claim 10, wherein the position sensor includes a remote sensor that is not connected to the front lens assembly, and wherein execution of the instructions causes the processor to receive the electronic position signal as an output signal from the remote sensor.

13. The control system of claim 12, wherein the remote sensor includes a camera, and wherein execution of the instructions causes the processor to receive the electronic position signal as image data from the camera.

14. The control system of claim 8, wherein the execution of the instructions causes the processor to receive an override signal from a human-machine interface (HMI) device in response to an operator input to the HMI device; and
interrupt the illumination switching control action in response to the override signal.

15. A method for controlling a wide angle viewing system (WAVS), comprising:
determining a position of a front lens assembly of the WAVS as one of an engaged position or a disengaged position; and
executing an illumination switching control action in response to the position of the front lens assembly, the illumination switching control action including:
turning off a microscope light source of a microscope and turning on an illumination tool when the front lens assembly is in the engaged position; and
turning on the microscope light source and turning off the illumination tool when the front lens assembly is in the disengaged position.

16. The method of claim 15, further comprising:
transmitting an electronic switching control signal to a surgical console to command the surgical console to turn the illumination tool on in the engaged position and off in the disengaged position.

17. The method of claim 15, further comprising:
receiving an electronic position signal from a position sensor, the electronic position signal being indicative of the position of the front lens assembly; and
determining whether the front lens assembly is in the engaged position or the disengaged position by processing the electronic position signal.

18. The method of claim 17, wherein receiving the electronic position signal includes receiving an encoder signal from an encoder.

19. The method of claim 17, wherein receiving the electronic position signal includes receiving an output signal from a remote sensor.

20. The method of claim 15, further comprising:
receiving an override signal from a human-machine interface (HMI) device in response to an operator input to the HMI device; and
interrupting the illumination switching control action in response to the override signal.
